(19) United States
(12) Patent Application Publication
(10) Pub. No.: US 2024/0325610 A1
(43) Pub. Date: Oct. 3, 2024

(54) DRUG COATING, DRUG-COATED BALLOON AND PREPARATION METHOD THEREOF

(71) Applicant: LEPU MEDICAL TECHNOLOGY (BEIJING) CO., LTD., Changping District, Beijing (CN)

(72) Inventors: Yangyang LIU, Changping District, Beijing (CN); Lixiao ZHAO, Changping District, Beijing (CN); Cuijie MA, Changping District, Beijing (CN); Yi ZHANG, Changping District, Beijing (CN); Xiaojun HU, Changping District, Beijing (CN)

(21) Appl. No.: 18/246,449

(22) PCT Filed: Jun. 10, 2022
(86) PCT No.: PCT/CN2022/098130

§ 371 Date: Mar. 23, 2023

(51) Int. Cl. A61L 29/16; A61L 29/08

(52) U.S. Cl. CPC A61L 29/16; A61L 29/085; A61L 2300/21; A61L 2300/22; A61L 2300/232; A61L 2300/30; A61L 2300/404; A61L 2300/41; A61L 2300/416; A61L 2300/426; A61L 2300/434; A61L 2400/12; A61L 2420/02; A61L 2420/06

(57) ABSTRACT

The present invention relates to the technical field of medical devices, in particular to a drug coating, a drug-coated balloon and a preparation method thereof. The drug coating comprises a drug active coating and a positively-charged hydrophobic modified layer which are sequentially attached to the surface of a substrate. By providing the positively charged hydrophobic modified layer comprising the positively-charged modified substance and the hydrophobic substance, the entire surface of the drug coating is given positive electric charge and hydrophobic property. Such positive electric charge characteristics make the drug coating can be strongly combined with the negatively-charged inner wall of blood vessels, so that the drug coating can be permanently adsorbed on the inner wall of blood vessels. The combination of positive electric charge characteristics and hydrophobic property can effectively resist the influence of blood flow on the erosion of drug coating during transportation.

### CROSS-REFERENCE TO RELATED APPLICATIONS

This application claims the benefit of International Application No. PCT/CN2022/098130, filed on Jun. 10, 2022. The entire disclosure of the above application is incorporated herein by reference.

### TECHNICAL FIELD

The present invention relates to the technical field of medical devices, in particular to a drug coating, a drug-coated balloon and a preparation method thereof.

### BACKGROUND

Coronary heart disease is the most common cause of death in the world today. The deaths number caused by coronary heart disease even exceeds the total death number caused by all kinds of cancers. According to incomplete statistics, the number of coronary heart disease patients in China has exceeded 11 million, and the number is increasing by 1 million every year. There were more than 900,000 percutaneous coronary intervention (PCI) operations in China in 2018. It is expected that PCI will continue to maintain an annual rate of 13%-16% growth in the next 3-5 years against a backdrop of younger age of coronary heart disease, an aging population and the continuous advancement of tiered diagnosis and treatment. Drug-coated balloon (DCB, referred to as drug balloon) is a coronary intervention technology developed in recent years. The surface of the drug balloon is coated with a drug coating, which is transferred to the blood vessel wall within a short dilation time after being delivered to the lesion site, and the drug coating releases anti-proliferative drugs to inhibit the proliferation of vascular smooth muscle cells. Drugs such as rapamycin are safe in the human body, but due to their poor lipophilicity, slow tissue absorption, and low bioavailability, the above shortcomings need to be overcome when applied to the field of drug balloons.

However, the current drug balloons mainly have the following problems: a large amount of drug is lost during the delivery process, and the blood wash or the dissolution and shedding of the drug coating causes a large loss of the drug coating on the surface of the balloon, thus affecting the drug amount transferred from the drug coating to the blood vessel wall of the lesions. According to research, 80% or more of the drugs in the drug-coated capsules in the prior art are lost in the drug delivery process, reducing the drug amount transferred to the blood vessel wall, thereby reducing the efficacy; These lost drugs may enter the human circulatory system and increase the risk of downstream vascular embolism, and increase the toxic and side effects of drug-coated balloons. Therefore, how to reduce the amount of drug delivered and increase the amount of drug transferred to the vascular wall to improve the efficacy is still an urgent problem for researchers to solve.

### SUMMARY OF THE INVENTION

Therefore, the technical problem to be solved by the present invention is to overcome the defect of low drug amount transferred from the drug balloon to blood vessel wall in the prior art, thereby providing a novel drug-coated balloon and a preparation method thereof.

To this end, the present invention provides a drug coating, wherein the drug coating comprises a drug active coating and a positively-charged hydrophobic modified layer which are sequentially attached to the surface of a substrate, and the positively-charged hydrophobic modified layer comprises a positively-charged modified substance and a hydrophobic substance.

In some embodiments, the drug active coating comprises a core-shell structure layer and/or a drug nanoparticle layer; wherein, the core-shell structure layer comprises a core-shell structure particle, the core-shell structure particle has an inner core and an outer shell surrounding the inner core, the inner core is a drug particle, and the outer shell is a polymer shell; and the drug nanoparticle layer comprises a drug nanoparticle.

In some embodiments, the core-shell structure particle has a particle size D50 of 100 nm to 9 m; preferably 300 nm to 6 m.

In some embodiments, the drug nanoparticle has a particle size D50 of 100 nm-600 nm.

In some embodiments, the drug coating comprises a core-shell structure layer, a drug nanoparticle layer and a positively-charged hydrophobic modified layer which are sequentially attached to the surface of the substrate.

In some embodiments, the drug coating has at least one feature selected from the group consisting of the following A-F:
A, the core-shell structure layer comprises a binder, preferably, the binder is at least one selected from the group consisting of polyvinyl alcohol, polyvinylpyrrolidone, Tween 80, poloxamer, egg yolk lecithin, soybean lecithin and methyl cellulose, preferably, a mass ratio of the binder to the core-shell structure particle is 1:1-26;
B, the polymer shell is an amphiphilic polymer shell, and the amphiphilic polymer comprises a hydrophilic block and a hydrophobic block, wherein the hydrophilic block is polyethylene glycol or polyethylene glycol monomethyl ether, and the hydrophobic block is at least one selected from the group consisting of polyoxypropylene, polystyrene, polyamino acid, polyglycolic acid, polylactide, polycaprolactone and poly(lactic-co glycolic acid); preferably, a mass ratio of the drug particle to the polymer shell in the core-shell structure particle is 0.5-5:0.5-50;
C, the drug nanoparticle layer comprises a binder, preferably, the binder is at least one selected from the group consisting of polyvinyl alcohol, polyvinylpyrrolidone, Tween 80, poloxamer, egg yolk lecithin, soybean lecithin and methyl cellulose; preferably, a mass ratio of the binder to the drug nanoparticle is 1:1-25;
D, a ratio of the drug content in the core-shell structure layer to the drug content in the drug nanoparticle layer is 1-5:1-5; preferably 1-2:1-2;
E, the drug is a medicine for preventing and treating coronary heart disease, preferably, the medicine for preventing and treating coronary heart disease is selected from rapamycin and/or a rapamycin derivative; and
F, the drug content in the core-shell structure layer is 1-5 μg/mm2; and/or the drug content in the drug nanoparticle layer is 1-5 μg/mm2.

Wherein, the polyamino acid in the present invention includes but is not limited to common polyamino acids such as polyglutamic acid, polyaspartic acid, and polyornithine.

In some embodiments, the drug nanoparticle layer is also provided with a positively-charged hydrophobic modified layer on the side away from the core-shell structure layer. Preferably, the positively-charged hydrophobic modified layer comprises a positively-charged lipid substance and a hydrophobic substance; more preferably, the positively-charged hydrophobic modified layer has at least one feature selected from the group consisting of following (1) to (3):
(1) a mass ratio of the positively-charged substance to the hydrophobic substance is 10-20:10-20, preferably 20:10;
(2) the hydrophobic substance is selected from fatty acids, natural phospholipids or synthetic phospholipids, preferably, the hydrophobic substance is at least one selected from the group consisting of nervonic acid, phytanic acid, palmitic acid, linolenic acid, caprylic acid, isooleic acid, stearic acid, palmitic acid, lauric acid, arachidonic acid, eicosapentaenoic acid, cephalin, lecithin, phosphatidylserine, phosphatidylglycerol, phosphatidylinositol, 1,2-dimyristoyl-sn-glycero-3-phospho-ethanolamine, 1,2-dipalmitoyl-rac-glycero-3-phosphocholine, distearoyl phosphatidyl choline, dilauroyl lecithin, and 1-stearoyl-2-myristoylphosphatidylcholine;
the positively-charged modified substance is a positively-charged lipid, preferably at least one selected from the group consisting of DC-cholesterol, dioleoyl phosphatidylethanolamine, 2-dioleoyl hydroxypropyl-3-N,N,N-trimethylammonium chloride, 1,2-di-O-octadecenyl-3-rimethylammonium propane, and (6Z,9Z,28Z,31Z)-heptatriacont-6,9,28,31-tetraene-19-yl 4-(dimethylamino)butanoate; more preferably, the hydrophobic substance is phytanic acid, and the positively-charged substance is dioleoyl phosphatidylethanolamine; and
(3) based on the total mass of the positively-charged modified substance and the hydrophobic substance, the content of the positively-charged hydrophobic modified layer is 0.1-1 μg/mm2.

In some embodiments, the core-shell structure particle comprises the following raw materials: 0.5-5 parts by weight of a drug, 0.5-50 parts by weight of an amphiphilic polymer, 1-50 parts by volume of an oil phase, 100-2000 parts by volume of an aqueous solution containing an emulsifier; wherein, the proportioning relationship between parts by weight and parts by volume is g/mL; optionally, the oil phase is selected from dichloromethane, acetone and any combination thereof.

In some embodiments, the drug nanoparticle comprises the following raw materials: 0.5-2 parts by weight of a drug, 5-20 parts by volume of an oil phase, and 5-100 parts by volume of an aqueous solution containing an emulsifier; wherein, the proportioning relationship between parts by weight and parts by volume is g/mL; optionally, the oil phase is selected from methanol, ethanol and any combination thereof.

In some embodiments, the emulsifier is at least one selected from the group consisting of polyvinyl alcohol, polyvinylpyrrolidone, poloxamer, bovine serum albumin, Tween 80, poloxamer, egg yolk lecithin, soybean lecithin and methyl cellulose; and/or, the emulsifier is present in an amount of 0.01-2% (weight/volume) in the aqueous solution containing the emulsifier.

The present invention also provides a drug-coated balloon, wherein the drug-coated balloon comprises a balloon body and a drug coating attached to the outside of the balloon body, wherein the drug coating is any of the above-mentioned drug coatings.

The present invention also provides a method for preparing a drug-coated balloon, comprising the following steps:
step S1: spraying a drug-containing coating solution on the outside of the balloon body, and drying to form a drug active coating;
step S2: mixing a positively-charged modified substance, a hydrophobic substance and an organic solvent to prepare a coating solution of the positively-charged hydrophobic modified layer, spraying the coating solution of the positively-charged hydrophobic modified layer on the outside of the drug active coating, and drying to form the positively-charged hydrophobic modified layer, obtaining a drug-coated balloon.

In the present invention, the drying method for forming the coating may be common drying such as airing and stoving.

In some embodiments, the organic solvent is at least one selected from the group consisting of alkanes, alcohols and water; and/or a ratio of the positively-charged modified substance by mass to the hydrophobic substance by mass to the organic solvent by volume is 10-20 mg:10-20 mg:5 mL. The exemplary organic solvent is at least one selected from n-heptane, n-hexane, n-pentane, methanol, ethanol and water.

In some embodiments, the drug-containing coating solution comprises a coating solution of the core-shell structure layer and/or a coating solution of the drug nanoparticle layer;

wherein the coating solution of the core-shell structure layer is prepared by: mixing the core-shell structure particle with the organic solvent to obtain the coating solution of the core-shell structure layer; and

wherein the coating solution of the drug nanoparticle layer is prepared by: mixing the drug nanoparticle with the organic solvent to obtain the coating solution of the drug nanoparticle layer.

In some embodiments, the organic solvent is at least one selected from the group consisting of alkanes, alcohols and water; and/or, in the coating solution of the core-shell structure layer, a ratio of the added core-shell structure particle by mass to the organic solvent by volume is 2.1 g:3-12 mL; and/or, in the coating solution of the drug nanoparticle layer, a ratio of the added drug nanoparticle by mass to the organic solvent by volume is 150 mg:3-12 mL. The exemplary of the organic solvent is at least one selected from n-heptane, n-hexane, n-pentane, methanol, ethanol and water.

The core-shell structure particle in the present invention can be prepared by conventional methods. An exemplary preparation is as follows: the drug and the amphiphilic polymer can be dissolved in an oil phase, then the oil phase is added dropwise to an aqueous solution containing an emulsifier, followed by performing at least one or a combination of high pressure homogenization, ultrasonic homogenization, solvent evaporation and membrane emulsification, then centrifugation, removal of supernatant, and drying to obtain the core-shell structure particle.

The drug nanoparticle in the present invention can be prepared by conventional methods. An exemplary preparation is as follows: the drug can be dissolved in an oil phase, then the oil phase is added dropwise to an aqueous solution containing an emulsifier, stirred or sonicated, and dried to obtain the drug nanoparticle.

The core-shell structure particle and the drug nanoparticle can be dried by conventional methods such as spray drying, freeze drying and vacuum drying. One or two lyoprotectants are added before freeze drying, types of which include but are not limited to mannitol, trehalose, sucrose, glucose, lactose, etc., and the lyoprotectant can be present in an amount of 0.1%-10% or 1-10% by mass in the drug.

The technical solution of the present invention has the following advantages:

1. The drug coating according to the present invention comprises a drug active coating and a positively-charged hydrophobic modified layer which are sequentially attached to the surface of a substrate, and the positively-charged hydrophobic modified layer comprises a positively-charged modified substance and a hydrophobic substance. By providing the positively charged hydrophobic modified layer comprising the positively-charged modified substance and the hydrophobic substance, the entire surface of the drug coating is given positive electric charge and hydrophobic property. Such positive electric charge characteristics make the drug coating can be strongly combined with the negatively-charged inner wall of blood vessels, so that the drug coating can be permanently adsorbed on the inner wall of blood vessels. The combination of positive electric charge characteristics and hydrophobic property can effectively resist the influence of blood flow on the erosion of drug coating during transportation. The drug coating with the above structure can not only reduce the loss of the drug during the delivery of the drug balloon, but also reduce the loss of the drug during balloon dilation and contraction, while increasing the amount of drug transferred to the inner wall of the blood vessel.

2. The drug coating according to the present invention is provided with the drug nanoparticle and/or the core-shell structure layer, in particular, the drug coating comprising a core-shell structure layer, a drug nanoparticle layer and a positively-charged hydrophobic modified layer which are sequentially attached to the surface of the substrate. On the one hand, this feature makes the drug nanoparticles more firmly bound on the surface of the balloon, not easy to fall off, thereby greatly reducing the amount of drug loss in the process of delivery in the body, increasing the amount of drug transferred to the blood vessel wall, and thus improving the curative effect; on the other hand, the drug nanoparticle is arranged in the drug nanoparticle layer and the drug particle is arranged in the core-shell structure layer, so that drugs in the drug nanoparticle layer can be released quickly after reaching the affected area to achieve the purpose of rapid onset, while drugs in the core-shell structure layer is slowly released and can achieve a sustained release effect and prolong the action time. Furthermore, it is possible to regulate the drug release by adjusting the drug content in the core-shell structure layer and the drug nanoparticle layer.

3. In the drug coating according to the present invention, the particle size D50 of the core-shell structure particles is controlled in a range of 100 nm and 9 m to regulate the drug to have a reasonable release period, and maintain the drug concentration in the target tissue at a relatively high therapeutic level. Especially controlling within a range of 300 nm to 6 m allows the drug to be released in about 90 days.

4. In the drug coating according to the present invention, the particle size of the drug nanoparticle is 100-600 nm, and the nano-scale drug particles can quickly penetrate into the target diseased tissue and act immediately.

5. In the drug coating provided in the present invention, the core-shell structure layer further comprises a binder which preferably is at least one selected from the group consisting of polyvinyl alcohol, polyvinylpyrrolidone, Tween 80, poloxamer, egg yolk lecithin, soybean lecithin and methyl cellulose; or the drug nanoparticle layer further comprises a binder which preferably is at least one selected from the group consisting of polyvinyl alcohol, polyvinylpyrrolidone, Tween 80, poloxamer, egg yolk lecithin, soybean lecithin and methyl cellulose; the addition of the binder increases the cohesive force between the core-shell particles, resulting in lower coating loss during transportation and dilation, increasing the affinity of the coating with the blood vessel wall, making the coating transfer higher; increasing the lipophilicity of the drug, allowing the drug to enter the target tissue faster.

6. The drug coating according to the present invention can further reduce the loss rate and increase the amount transferred to vessel wall by adopting the hydrophobic substances and positively-charged modified substances listed in the present invention, especially by selecting phytanic acid as the hydrophobic substance and dioleoyl phosphatidylethanolamine as the positively-charged modified substance.

### BRIEF DESCRIPTION OF THE DRAWINGS

In order to illustrate the specific embodiments of the present invention or the technical solutions in the prior art more clearly, the following briefly introduces the accompanying drawings that need to be used in the description of the specific embodiments or the prior art. Obviously, the accompanying drawings in the following description are some embodiments of the present invention. For those of ordinary skill in the art, other drawings can also be obtained based on these drawings without creative efforts.

FIG. 1 is a scanning electron microscope image of the core-shell structure particle prepared in Example 1 of the present invention;

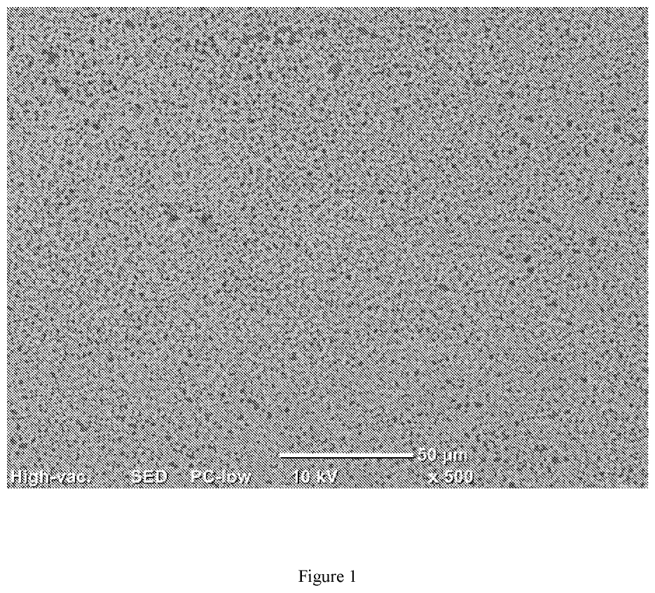

FIG. 2 is a scanning electron microscope image of the drug nanoparticle prepared in Example 1 of the present invention;

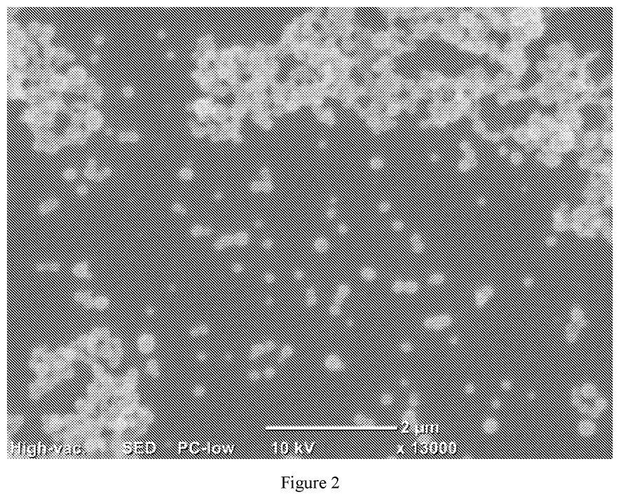

FIG. 3 is a scanning electron microscope image of the drug balloon prepared in Example 1 of the present invention.

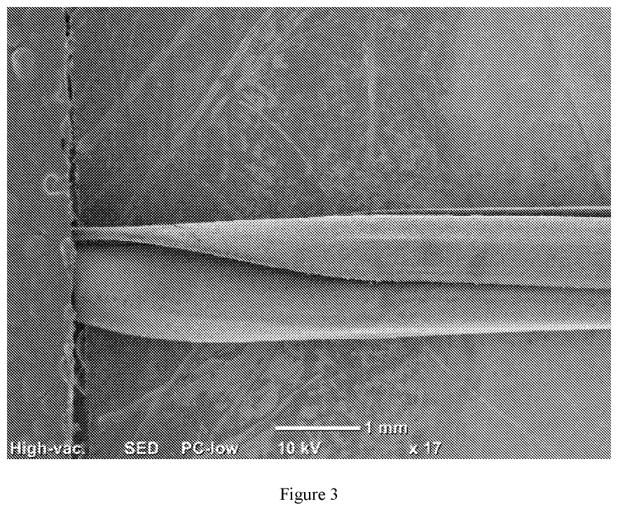

### DETAILED DESCRIPTION

The following embodiments are provided for a better understanding of the present invention but not for limiting the content and protection scope of the present invention. Any product identical or similar to the present invention obtained by combining with the features of other prior art shall fall within the protection scope of the present invention.

If the specific experimental steps or conditions are not indicated in the examples, it can be carried out according to the operations or conditions of the conventional experimental steps described in the literature in this field. The reagents or instruments used without the manufacturer's indication are all conventional reagent products that can be obtained from commercial sources.

For example, the polyethylene glycol-polycaprolactone block copolymer (PEG-PCL) used in the present invention was purchased from SHANGHAI ZZBIO CO., LTD., PEG number average molecular weight: 2000, PCL number average molecular weight: 2000. Polyethylene glycol-polylactide block copolymer (PEG-PLA) was purchased from SHANGHAI ZZBIO CO., LTD., PEG number average molecular weight: 2000, PLA viscosity: 0.9-1.2 dl/g. Polyethylene glycol-polyglycolide lactide block copolymer (PEG-PLGA), PEG number average molecular weight: 2000, PLGA (75:25) viscosity: 0.5-0.8 dl/g, PLGA (50:50) viscosity: 0.3-0.5 dl/g, all purchased from: XI'AN RUIXI BIOLOGICAL TECHNOLOGY CO., LTD.

Polyvinyl alcohol (PVA) was purchased from GUANGZHOU STANDARD PHARMA LTD., model: 4-88; Sodium alginate was purchased from QINGDAO HUANGHAI BIO-PHARMACEUTICAL CO., LTD.; Trehalose was purchased from JIANGSU HI-STONE PHARMACEUTICAL CO., LTD., Specification: injection grade; Poloxamer 188, was purchased from SIGMA-ALDRICH (SHANGHAI) TRADING CO., LTD.; Lactose was purchased from JIANGSU HI-STONE PHARMACEUTICAL CO., LTD. Specification: injection grade. The egg yolk lecithin was purchased from Lipoid Company, specification: injection grade; the balloon was provided by LEPU MEDICAL TECHNOLOGY (BEIJING) CO., LTD., 3.5 mm diameter×20 mm length.

The method for testing the drug loading of the core-shell structured particles in the present invention is as follows: 50 mg of the core-shell structured particles is mixed with 5 mL of acetonitrile, sonicated for 10 minutes, diluted with acetonitrile to 10 mL, and centrifuged. The supernatant is collected and injected to a high performance liquid chromatograph respectively to determine the content of the drug (rapamycin) in core-shell structured particles according to the Pharmacopoeia of the People's Republic of China (2020 edition) general rule 0512 high performance liquid chromatography, with octadecylsilane bonded silica gel as a filler, methanol-acetonitrile-water (60:17:23) as a mobile phase, under a detection wavelength of 280 nm, at a flow rate of 1 mL/min, and a column temperature of 40° C. Particle size D50 test: 10 mg of core-shell structured particles or drug nanoparticles is dispersed well in 5 ml of purified water, and then tested with a particle size distribution analyzer; the average value of three consecutive measurements is taken.

### Example 1

A drug-coated balloon was provided in this embodiment. The drug-coated balloon comprised a balloon body, and a core-shell structure layer, a drug nanoparticle layer and a positively-charged hydrophobic modified layer which were sequentially attached to the outside of the balloon body, wherein the core-shell structure layer comprised a core-shell structure particle, the core-shell structure particle had an inner core and an outer shell surrounding the inner core, the inner core was a rapamycin particle, and the outer shell was a PEG-PCL shell; the drug nanoparticle layer comprised rapamycin nanoparticles as drug nanoparticles. The positively-charged hydrophobic modified layer comprised a positively-charged modified substance (DC-cholesterol) and a hydrophobic modified substance (stearic acid).

The preparation method of the above-mentioned drug-coated balloon comprised the following steps:

### (1) Preparation of Core-Shell Structure Particles

1 g of rapamycin and 2 g of PEG-PCL were weighed and dissolved in 30 mL of an oil phase (dichloromethane and acetone at a volume ratio of 8:2). Under stirring (normal temperature, 1000 r/min), 200 ml of purified water containing 0.1% (w/v) PVA was dropwise (1 mL/min) injected, stirred for 12 h (35° C., 750r/min), and centrifuged at −4° C. and 12000r/min for 30 min. The supernatant was removed, and the bottom precipitate was washed for twice with 200 ml of purified water each time, centrifuged, and freeze-dried to obtain core-shell structure particles. Test showed that the core-shell particles had a particle diameter D50 of 312 nm, and a drug loading of 6.83%.

### (2) Preparation of Drug Nanoparticles

Rapamycin drug nanoparticles were prepared by anti-solvent precipitation method. 0.5 g of rapamycin was weighed and dissolved in 5 ml of methanol to obtain an oil phase. The oil phase was added dropwise to 15 ml of an aqueous solution containing 2% (w/v) of poloxamer 188 and sonicated for 30 min, then 25 mg of trehalose was added thereto and dispersed well, then freeze-dried to obtain drug nanoparticles. After testing, the particle size D50 of the drug nanoparticles was 481 nm.

### (3) Preparation of Coating Solution

2.1 g of core-shell structure particles and 83.6 mg of egg yolk lecithin were added to 12 ml of ethanol and dispersed well by ultrasonic to obtain a coating solution of core-shell structure layer. 150 mg of drug nanoparticles and 7.1 mg of egg yolk lecithin were added to 12 ml of ethanol and dispersed well by ultrasonic to obtain a coating solution of drug nanoparticle layer. 20 mg of DC-cholesterol and 20 mg of stearic acid were added into 5 ml of ethanol and dispersed well by ultrasonic to obtain a coating solution of a positively-charged hydrophobic modified layer.

### (4) Preparation of Drug Balloon

The first spraying: the coating solution of the core-shell structure layer was sprayed on the surface of the balloon by using a balloon spraying machine at following spraying parameters of: a dripping flow rate: 3.6 ml/h, an ultrasonic power: 0.05 W, so that the concentration of rapamycin on the surface of the balloon reached 1.0 μg/mm², and it was air-dried;

The second spraying: the coating solution of the drug nanoparticle layer was sprayed on the surface of the balloon by using a balloon spraying machine at following spraying parameters of: a dripping flow rate: 3.6 ml/h, an ultrasonic power: 0.05 W, so that the concentration of rapamycin on the surface of the balloon reached 2.0 μg/mm², and it was air-dried;

The third spraying: the coating solution of the positively-charged hydrophobic modified layer was sprayed on the surface of the balloon by using a balloon spraying machine at following spraying parameters of: drip flow rate: 3.6 ml/h, ultrasonic power: 0.05 W, and it was air-dried. The balloon after the third spraying had an average weight gain of 0.2 μg/mm². The average weight gain of the third spraying balloon in the present invention was calculated as follows: the average weight gain of the third spraying balloon=(the weight of the balloon after the third spraying and drying—the weight of the balloon after the second spraying and drying)/balloon surface area.

### Example 2

A drug-coated balloon and a preparation method thereof provided in this embodiment differed from Example 1 only in that the composition and preparation method of the coating solution of the positively-charged hydrophobic modified layer were different. In this embodiment, 20 mg of DC-cholesterol and 10 mg of stearic acid were added to 5 ml of ethanol and dispersed well by ultrasonic to obtain a coating solution of a positively-charged hydrophobic modified layer.

### Example 3

A drug-coated balloon and a preparation method thereof provided in this embodiment differed from Example 1 only in that the average weight gain of the balloon after the third spraying was 0.1 μg/mm².

### Example 4

A drug-coated balloon and a preparation method thereof provided in this embodiment differed from Example 1 only in that the composition and preparation method of the coating solution of the positively-charged hydrophobic modified layer were different and the weight gain by spraying was different. In this embodiment, 20 mg of dioleoyl phosphatidylethanolamine (DOPE) and 20 mg of phytanic acid were added into 5 ml of ethanol and dispersed well by ultrasonic to obtain a coating solution of a positively-charged hydrophobic modified layer for later use. In this embodiment, the average weight gain of the balloon after the third spraying was 0.1 μg/mm².

### Example 5

A drug-coated balloon and a preparation method thereof provided in this embodiment differed from Example 1 only in that the composition and preparation method of the coating solution of the positively-charged hydrophobic modified layer were different. In this embodiment, 20 mg of DOPE and 10 mg of phytanic acid were added to 5 ml of ethanol and dispersed well by ultrasonic to obtain a coating solution of a positively-charged hydrophobic modified layer for later use.

### Example 6

A drug-coated balloon and a preparation method thereof provided in this embodiment differed from Example 1 only in that the composition and preparation method of the coating solution of the positively-charged hydrophobic modified layer were different. In this embodiment, 20 mg of DOPE and 20 mg of phytanic acid were added to 5 ml of ethanol and dispersed well by ultrasonic to obtain a coating solution of a positively-charged hydrophobic modified layer for later use.

### Example 7

A drug-coated balloon and a preparation method thereof provided in this embodiment differed from Example 1 only in that the preparation methods of the coating solution of the core-shell structure layer and the coating solution of the drug nanoparticle layer were different. In this embodiment, 2.1 g of the core-shell structure particles were added into 12 ml of ethanol and dispersed well by ultrasonic to obtain a coating solution of the core-shell structure layer. 150 mg drug nanoparticles were added into 12 ml of ethanol and dispersed well by ultrasonic to obtain a coating solution of the drug nanoparticle layer.

### Example 8

A drug-coated balloon was provided in this embodiment. The drug-coated balloon comprised a balloon body, and a core-shell structure layer, a drug nanoparticle layer and a positively-charged hydrophobic modified layer which were sequentially attached to the outside of the balloon body, wherein the core-shell structure layer comprised a core-shell structure particle, the core-shell structure particle had an inner core and an outer shell surrounding the inner core, the inner core was a rapamycin particle, and the outer shell was a PEG-PLA shell; the drug nanoparticle layer comprised rapamycin nanoparticles as drug nanoparticles. The positively-charged hydrophobic modified layer comprised DC-cholesterol and stearic acid.

The preparation method of the above-mentioned drug-coated balloon comprised the following steps:

### (1) Preparation of Core-Shell Structure Particles

0.5 g of rapamycin and 0.5 g of PEG-PLA were weighed and dissolved in 1 mL of oil phase (acetone). Under stirring (normal temperature, 1000r/min), 100 ml of purified water containing 0.1% (w/v) PVA was dropwise (1 mL/min) injected, stirred for 12 h (35° C., 750r/min), centrifuged at −4° C. and 12000r/min for 30 min. The supernatant was removed, and the bottom precipitate was washed for twice with 200 ml of purified water each time, centrifuged, and freeze-dried to obtain core-shell structure particles. Test showed that the core-shell particles had a particle diameter D50 of was 579 nm and a drug loading of 5.92%.

### (2) Preparation of Drug Nanoparticles

Rapamycin drug nanoparticles were prepared by anti-solvent precipitation method. 2 g of rapamycin was weighed and dissolved in 20 ml of ethanol to obtain an oil phase. The oil phase was added dropwise to 100 ml of an aqueous solution containing 2% (w/v) of poloxamer 188 and sonicated for 30 min, then 25 mg of mannitol was added thereto and dispersed well, then freeze-dried to obtain drug nanoparticles. After testing, the particle size D50 of the drug nanoparticles was 593 nm.

### (3) Preparation of Coating Solution

2.1 g of core-shell structure particles and 2.1 g of Twain 80 were added to 3 ml of water and dispersed well by ultrasonic to obtain a coating solution of core-shell structure layer. 150 mg of drug nanoparticles and 150 mg of poloxamer 188 were added to 3 ml of water and dispersed well by ultrasonic to obtain a coating solution of drug nanoparticle layer. 20 mg of DC-cholesterol and 20 mg of stearic acid were added into 5 ml of n-hexane and dispersed well by ultrasonic to obtain a coating solution of a positively-charged hydrophobic modified layer.

### (4) Preparation of Drug Balloon

The first spraying: the coating solution of the core-shell structure layer was sprayed on the surface of the balloon by using a balloon spraying machine at following spraying parameters of: the dripping flow rate: 3.6 ml/h, the ultrasonic power: 0.05 W, so that the concentration of rapamycin on the surface of the balloon reached 1.0 μg/mm², and it was air-dried;

The second spraying: the coating solution of the drug nanoparticle layer was sprayed on the surface of the balloon by using a balloon spraying machine at following spraying parameters of: the dripping flow rate: 3.6 ml/h, the ultrasonic power: 0.05 W, so that the concentration of rapamycin on the surface of the balloon reached 2.0 μg/mm², and it was air-dried;

The third spraying: the coating solution of the positively-charged hydrophobic modified layer was sprayed on the surface of the balloon by using a balloon spraying machine at following spraying parameters of: drip flow rate: 3.6 ml/h, ultrasonic power: 0.05 W, and it was air-dried. The balloon after the third spraying had an average weight gain of 0.2 μg/mm².

### Example 9

A drug-coated balloon and a preparation method thereof were provided in this embodiment. The drug nanoparticles, the coating solution of drug nanoparticle layer and the coating solution of positively-charged hydrophobic modification layer were prepared according to the methods of Example 1.

The first spraying: the coating solution of the drug nanoparticle layer was sprayed on the surface of the balloon by using a balloon spraying machine at following spraying parameters: the dripping flow rate: 3.6 ml/h, the ultrasonic power: 0.05 W, so that the concentration of rapamycin on the surface of the balloon reached 2.0 μg/mm², and it was air-dried;

The second spraying: the coating solution of the positively-charged hydrophobic modified layer was sprayed on the surface of the balloon by using a balloon spraying machine at following spraying parameters: drip flow rate: 3.6 ml/h, ultrasonic power: 0.05 W, and it was air-dried. The balloon after the second spraying had an average weight gain of 0.2 μg/mm². The average weight gain of the balloon after the second spraying in the present invention was calculated as follows: the average weight gain of the second spraying balloon=(the weight of the balloon after the second spraying and drying—the weight of the balloon after the first spraying and drying)/balloon surface area.

### Example 10

A drug-coated balloon and a preparation method thereof were provided in this embodiment. The core-shell structure particles, the coating solution of the core-shell structure layer and the coating solution of positively-charged hydrophobic modification layer were prepared according to the method of Example 1.

The first spraying: the coating solution of the core-shell structure layer was sprayed on the surface of the balloon by using a balloon spraying machine at following spraying parameters of: the dripping flow rate: 3.6 ml/h, the ultrasonic power: 0.05 W, so that the concentration of rapamycin on the surface of the balloon reached 2.0 μg/mm², and it was air-dried;

The second spraying: the coating solution of the positively-charged hydrophobic modified layer was sprayed on the surface of the balloon by using a balloon spraying machine at following spraying parameters: drip flow rate: 3.6 ml/h, ultrasonic power: 0.05 W, and it was air-dried. The balloon after the second spraying had an average weight gain of 0.2 μg/mm².

### Comparative Example 1

A drug-coated balloon was provided in this comparative example. The drug-coated balloon comprised a balloon body, and a core-shell structure layer and a drug nanoparticle layer which were sequentially attached to the outside of the balloon body, wherein the core-shell structure layer comprised a core-shell structure particle, the core-shell structure particle had an inner core and an outer shell surrounding the inner core, the inner core was a rapamycin particle, and the outer shell was a PEG-PCL shell; the drug nanoparticle layer comprised rapamycin nanoparticles as drug nanoparticles.

The above-mentioned drug-coated balloon was prepared according to the method of Example 1, except that the surface of the balloon was not sprayed for the third time, that is, no coating solution of the positively-charged hydrophobic modified layer was sprayed.

### Experimental Example 1

### Investigating the In Vitro Drug Release of Core-Shell Structure Particles with Different Particle Sizes

To investigate the in vitro release of core-shell structure particles with different particle sizes, 5 groups of experiments were done. The mass of rapamycin and PEG-PCL, the volume of oil phase, and the volume ratio of dichloromethane to acetone in the oil phase and the stirring speed for each group of experiments are shown in the following Table 1. Rapamycin and PEG-PCL according to the following table prescription were weighed respectively, dichloromethane and acetone were weighed according to the prescription table below and mixed to prepare the oil phase, then rapamycin and PEG-PCL were dissolved in the oil phase, resulting in a drug-containing oil phase. Then the drug-containing oil phase was stirred at the corresponding rotation speed in the table at normal temperature, and 200 ml of an aqueous solution containing 0.1% (w/v) PVA was dropwise (1 mL/min) injected under stirring. After injecting was completed, the stirring was continued at 750 r/min for 12 h at a temperature of 35° C. Then, centrifugation was carried out at a rotating speed of 12000 r/min and a temperature of −4° C. for 30 min, the supernatant was removed, the bottom precipitate was washed twice with purified water, 200 ml of water each time, centrifuged, and freeze-dried to obtain a core-shell structure particle.

TABLE 1
Core-shell structure particles and particle sizes thereof
obtained with different formulations and process conditions

| | | | | Volume ratio | | |
|---|---|---|---|---|---|---|
| | | | | of Methylene | | |
| | | | | Chloride to | | |
| Experimental | | | Oil | Acetone in | Speed of | Particle |
| Group No. | Rapamycin/g | PEG-PCL/g | Phase/mL | the Oil Phase | Stirring/rpm | Size D50 |
| Experimental | 0.5 | 1.0 | 50 | 8:2 | 1000 | 108 nm |
| Group 1 | | | | | | |
| Experimental | 1.0 | 2.0 | 30 | 8:2 | 700 | 341 nm |
| Group 2 | | | | | | |
| Experimental | 1.0 | 2.0 | 15 | 8:2 | 600 | 2.59 μm |
| Group 3 | | | | | | |
| Experimental | 1.0 | 3.0 | 15 | 8:2 | 600 | 5.94 μm |
| Group 4 | | | | | | |
| Experimental | 1.0 | 4.0 | 10 | 8:2 | 400 | 8.73 μm |
| Group 5 | | | | | | |

Then the in vitro drug release of the core-shell structure particles was tested by the following method.

10 mg of core-shell structure particles were mixed with an appropriate amount of a release medium, shaken to disperse well, fully transferred into a 1000 ml volumetric flask, diluted with the release medium to the mark, and shaken to mix well. Shake flask method was carried out as follows: 160 rpm/min, release medium was PBS (pH7.4) solution containing 0.0300 (w/v) SDS, volume was 40 ml; samples were taken at: 1 day, 4 days, 7 days, 14 days, 28 days, 60 days and 90 days respectively, 1.5 ml for each sample, and 1.5 ml replenishment after taking of each sample. The solution to be tested was passed through a 0.5 um filter membrane for later use. The content of rapamycin in the test solution was determined according to General Chapter 0512 of the Pharmacopoeia of the People's Republic of China (2020 edition), using octadecylsilane-bonded silica gel as the filler, methanol-acetonitril e-water (at a volume ratio of 60:17:23) as the mobile phase, under a detection wavelength of 280 nm, with a flow rate of 1 mL/min and a column temperature of 40° C.

TABLE 2
Cumulative release (%)

| Sampling | Experi- | Experi- | Experi- | Experi- | Experi- |
|---|---|---|---|---|---|
| Time | mental | mental | mental | mental | mental |
| (Day) | Group 1 | Group 2 | Group 3 | Group 4 | Group 5 |
| 1 | 30.2 | 13.2 | 18.8 | 14.3 | 15.8 |
| 4 | 37.9 | 30.2 | 28.4 | 20.1 | 18.3 |
| 7 | 49.3 | 40.8 | 39.3 | 25.4 | 20.1 |
| 14 | 72.1 | 62.4 | 49.2 | 30.2 | 25.4 |
| 28 | 100 | 78.2 | 58.3 | 41.5 | 30.9 |
| 60 | No Test | 91.6 | 88.6 | 70.4 | 57.2 |
| 90 | No Test | 100 | 100 | 84.2 | 69.9 |

It can be seen from the above table that, compared with the experimental group 1, the experimental groups 2-4 had better sustained-release effect, and the effect of prolonging the action time of the drug was better. However, in test group 1, the drug release was faster due to the smaller particle size, and 100% of the drug was released within 28 days, while the drug release cycle in test group 5 was too long due to the larger particle size, and only 69.9% was released in 90 days. In the present invention, the particle size D50 of the core-shell structure particles was preferably 300 nm to 6 m.

### Experimental Example 2

In vitro simulation transport equipment (refer to the pharmaceutical industry standard “YY/T 0807-2010”) was used to simulate in vivo delivery withdrawal and dilation of the drug balloons prepared by each example and the comparative example to study drug loss during delivery, withdrawal and dilation and the drug coating transferring to the inner wall of the blood vessel during dilation, thereby indirectly evaluating the effectiveness, safety and anti-scour ability of the coating.

Test method: The in vitro simulated transport equipment was heated in a water bath and maintained at a temperature of 37° C. The end of the equipment was connected to a silicone simulated blood vessel (provided by Preclinic Medtech (Shanghai) Co., Ltd., diameter 3.0 mm). The drug balloon product was inserted into this equipment. When the balloon body entered the simulated blood vessel, the balloon was inflated to a pressure of 8 atm. After maintaining the pressure for 1 min, negative pressure was applied. Liquids in the simulated blood vessel, the simulated blood vessel, liquids in the in vitro simulated transportation equipment and the balloon after dilation were collected in turn. The content of rapamycin therein was tested according to the following method. The sample to be tested was dissolved in solvent acetonitrile, quantitatively diluted, and centrifuged. The supernatant was taken and injected into a liquid chromatograph. The content of rapamycin in the test solution was determined according to General Chapter 0512 of the Pharmacopoeia of the People's Republic of China (2020 edition), using octadecylsilane-bonded silica gel as the filler, methanol-acetonitrile-water (at a volume ratio of 60:17:23) as the mobile phase, under the detection wavelength of 280 nm, with a flow rate of 1 mL/min and a column temperature of 40° C. The chromatogram was recorded, the total mass of rapamycin in the liquid in the simulated blood vessel, simulated blood vessel, the liquid in the in vitro simulated transportation equipment and the balloon after dilation were calculated, and the percentage of rapamycin in each part with respect to the total mass was calculated.

The results were shown in Table 3.

TABLE 3
Experimental results of in vitro simulated transport

| | Residual | Loss | Transferred | Loss during |
|---|---|---|---|---|
| | drug | during | amount to | balloon |
| | of the | transpor- | vessel | dilation and |
| Group | balloon/% | tation/% | wall/% | contraction/% |
| Example 1 | 25.29% | 13.05% | 33.06% | 28.60% |
| Example 2 | 26.28% | 10.81% | 35.45% | 27.46% |
| Example 3 | 30.26% | 11.32% | 31.74% | 26.68% |
| Example 4 | 20.26% | 15.65% | 40.77% | 23.32% |
| Example 5 | 21.27% | 14.46% | 44.16% | 20.11% |
| Example 6 | 23.29% | 14.05% | 43.06% | 19.60% |
| Example 7 | 18.81% | 19.63% | 30.15% | 31.41% |
| Example 8 | 25.84% | 20.61% | 31.72% | 21.83% |
| Example 9 | 18.69% | 20.12% | 30.44% | 30.75% |
| Example 10 | 15.84% | 21.58% | 30.19% | 32.39% |
| Comparative | 9.47% | 53.14% | 7.22% | 30.17% |
| Example 1 | | | | |

The results in the above table showed that, compared with Comparative Example 1, the amount of drug transferred to the blood vessel wall by the drug coating of Examples 1-10 of the present invention was significantly increased, and the amount of loss during transportation was significantly reduced.

The comparison between Example 1 and Example 7 showed that, in the preferred solution of the present invention, the amount of the drug transferred to the blood vessel wall can be further increased by adding a binder to the core-shell structure layer and adding a binder to the nanoparticle layer, and losses during the delivery process and losses during balloon dilation-contraction were reduced.

The comparison between Example 1 and Examples 9-10 showed that, in the preferred solution of the present invention, the core-shell structure layer, the drug nanoparticle layer and the positively-charged hydrophobic modified layer are sequentially attached to the surface of the substrate, as a result the amount of drug transferred to the blood vessel wall can be further increased, and the loss during delivery and the loss during balloon dilation-contraction were reduced.

The comparison of Examples 1-6 showed that in the preferred solution of the present invention, by using phytanic acid and dioleoyl phosphatidylethanolamine as the hydrophobic substance and the positively-charged modified substance, the amount of the drug transferred to the blood vessel wall can be further increased, and the loss during delivery and the loss during balloon dilation-contraction were reduced.

The comparison between Examples 1-3 and Examples 4-6 showed that, in the present invention, the amount of the drug transferred to the blood vessel wall can be further increased by controlling the mass ratio of the positively-charged modified substance to the hydrophobic substance within a preferred range.

### Experimental Example 3

### In Vivo Pharmacokinetics Experiment

Twelve white pigs were randomly divided into 2 groups, an experimental group and a control group, with 6 white pigs in each group. The experimental group white pigs received 1 interventional drug balloon of Example 5 in each of coronary vessels LAD (left anterior descending branch), LCX (left circumflex branch) and RCA (right coronary artery) respectively, and the control group white pigs received 1 interventional drug balloon of Comparative Example 1 in each of coronary vessels LAD (left anterior descending branch), LCX (left circumflex branch) and RCA (right coronary artery). Each balloon was dilated once for 60 S, and withdrawn. Then the animals in the experimental group and the control group were sacrificed at 0 days (post-intervention), 7 days, and 28 days, respectively. 2 animals in each group were sacrificed at each time point. Blood vessels undergoing balloon dilation were collected and stored at −80° C. The blood vessel wall tissue samples were thawed at room temperature, and the average concentration of rapamycin in the blood vessel tissue samples was tested by LC-MS/MS method. The results were shown in the following table.

TABLE 4
Drug concentration/(ng/mg) in blood vessel tissue

| | 0 day | 7 days | 28 days |
|---|---|---|---|
| Experimental Group | 215.44 | 59.61 | 6.20 |

The results of the pharmacokinetic study showed that the amount of the drug released into the vascular tissue from the drug balloon prepared in Example 5 of the present invention was significantly higher than that of the drug balloon of the comparative example.

Obviously, the above-mentioned embodiments are only examples for clear description, and are not intended to limit the implementation manner. For those of ordinary skill in the art, changes or modifications in other different forms can also be made on the basis of the above description. There is no need and cannot be exhaustive of all implementations here. And the obvious changes or changes derived from this are still within the protection scope of the present invention.

1-15. (canceled)

16. A drug coating, wherein the drug coating comprises a drug active coating and a positively-charged hydrophobic modified layer which are sequentially attached to the surface of a substrate, and the positively-charged hydrophobic modified layer comprises a positively-charged modified substance and a hydrophobic substance.

17. The drug coating of claim 16, wherein the drug active coating comprises a core-shell structure layer and/or a drug nanoparticle layer; wherein,
the core-shell structure layer comprises a core-shell structure particle, the core-shell structure particle has an inner core and an outer shell surrounding the inner core, the inner core is a drug particle, and the outer shell is a polymer shell; and
the drug nanoparticle layer comprises a drug nanoparticle.

18. The drug coating of claim 17, wherein the core-shell structure particle has a particle size D50 of 100 nm to 9 m, preferably 300 nm to 6 m.

19. The drug coating of claim 17, wherein the drug nanoparticle has a particle size D50 of 100 nm to 600 nm.

20. The drug coating of claim 17, wherein the drug coating comprises a core-shell structure layer, a drug nanoparticle layer and a positively-charged hydrophobic modified layer which are sequentially attached to the surface of the substrate.

21. The drug coating of claim 17, wherein the drug coating has at least one feature selected from the group consisting of A to F:
A, the core-shell structure layer comprises a binder, preferably, the binder is at least one selected from the group consisting of polyvinyl alcohol, polyvinylpyrrolidone, Tween 80, poloxamer, egg yolk lecithin, soybean lecithin and methyl cellulose, preferably, a mass ratio of the binder to the core-shell structure particle is 1:1-26;
B, the polymer shell is an amphiphilic polymer shell, and the amphiphilic polymer comprises a hydrophilic block and a hydrophobic block, wherein the hydrophilic block is polyethylene glycol or polyethylene glycol monomethyl ether, and the hydrophobic block is at least one selected from the group consisting of polyoxypropylene, polystyrene, polyamino acid, polyglycolic acid, polylactide, polycaprolactone and poly(lactic-co glycolic acid); preferably, a mass ratio of the drug particle to the polymer shell in the core-shell structure particle is 0.5-5:0.5-50;
C, the drug nanoparticle layer comprises a binder, preferably, the binder is at least one selected from the group consisting of polyvinyl alcohol, polyvinylpyrrolidone, Tween 80, poloxamer, egg yolk lecithin, soybean lecithin and methyl cellulose; preferably, a mass ratio of the binder to the drug nanoparticle is 1:1-25;
D, a ratio of the drug content in the core-shell structure layer to the drug content in the drug nanoparticle layer is 1-5:1-5; preferably 1-2:1-2;
E, the drug is a medicine for preventing and treating coronary heart disease, preferably, the medicine for preventing and treating coronary heart disease is selected from rapamycin and/or a rapamycin derivative; and
F, the drug content in the core-shell structure layer is 1-5 μg/mm²; and/or the drug content in the drug nanoparticle layer is 1-5 μg/mm².

22. The drug coating of claim 16, wherein the positively-charged hydrophobic modified layer has at least one feature selected from the group consisting of following (1) to (3):
(1) a mass ratio of the positively-charged substance to the hydrophobic substance is 10-20:10-20, preferably 20:10;
(2) the hydrophobic substance is selected from fatty acids, natural phospholipids or synthetic phospholipids, preferably, the hydrophobic substance is at least one selected from the group consisting of nervonic acid, phytanic acid, palmitic acid, linolenic acid, caprylic acid, isooleic acid, stearic acid, palmitic acid, lauric acid, arachidonic acid, eicosapentaenoic acid, cephalin, lecithin, phosphatidylserine, phosphatidylglycerol, phosphatidylinositol, 1,2-dimyristoyl-sn-glycero-3-phospho-ethanolamine, 1,2-dipalmitoyl-rac-glycero-3-phosphocholine, distearoyl phosphatidyl choline, dilauroyl lecithin, and 1-stearoyl-2-myristoylphosphatidylcholine;
the positively-charged modified substance is a positively-charged lipid, preferably at least one selected from the group consisting of DC-cholesterol, dioleoyl phosphatidylethanolamine, 2-dioleoyl hydroxypropyl-3-N,N,N-trimethylammonium chloride, 1,2-di-O-octadecenyl-3-rimethylammonium propane, and (6Z,9Z,28Z,31Z)-heptatriacont-6,9,28,31-tetraene-19-yl 4-(dimethylamino)butanoate;
more preferably, the hydrophobic substance is phytanic acid, and the positively-charged substance is dioleoyl phosphatidylethanolamine; and
(3) based on the total mass of the positively-charged modified substance and the hydrophobic substance, the content of the positively-charged hydrophobic modified layer is 0.1-1 g/mm².

23. The drug coating of claim 17, wherein the core-shell structure particle comprises the following raw materials: 0.5-5 parts by weight of a drug, 0.5-50 parts by weight of an amphiphilic polymer, 1-50 parts by volume of an oil phase, 100-2000 parts by volume of an aqueous solution containing an emulsifier; wherein, the proportioning relationship between parts by weight and parts by volume is g/mL; optionally, the oil phase is selected from dichloromethane, acetone and any combination thereof.

24. The drug coating of claim 17, wherein the drug nanoparticle comprises the following raw materials: 0.5-2 parts by weight of a drug, 5-20 parts by volume of an oil phase, and 5-100 parts by volume of an aqueous solution containing an emulsifier; wherein, the proportioning relationship between parts by weight and parts by volume is g/mL; optionally, the oil phase is selected from methanol, ethanol and any combination thereof.

25. The drug coating of claim 24, wherein the emulsifier is at least one selected from the group consisting of polyvinyl alcohol, polyvinylpyrrolidone, poloxamer, bovine serum albumin, Tween 80, poloxamer, egg yolk lecithin, soybean lecithin and methyl cellulose; and/or, the emulsifier is present in an amount of 0.01-2% (weight/volume) in the aqueous solution containing the emulsifier.

26. A drug-coated balloon, comprising a balloon body and a drug coating of claim 16 attached to the outside of the balloon body.

27. A method for preparing a drug-coated balloon of claim 26, comprising the steps of:
step S1: spraying a drug-containing coating solution on the outside of the balloon body, and drying to form a drug active coating;
step S2: mixing a positively-charged modified substance, a hydrophobic substance and an organic solvent to prepare a coating solution of the positively-charged hydrophobic modified layer, spraying the coating solution of the positively-charged hydrophobic modified layer on the outside of the drug active coating, and drying to form the positively-charged hydrophobic modified layer, obtaining a drug-coated balloon.

28. The method for preparing a drug-coated balloon of claim 27, wherein the organic solvent is at least one selected from the group consisting of alkanes, alcohols and water; and/or a ratio of the positively-charged modified substance by mass to the hydrophobic substance by mass to the organic solvent by volume is 10-20 mg:10-20 mg:5 mL.

29. The method for preparing a drug-coated balloon of claim 27, wherein the drug-containing coating solution comprises a coating solution of the core-shell structure layer and/or a coating solution of the drug nanoparticle layer;
wherein the coating solution of the core-shell structure layer is prepared by: mixing the core-shell structure particle with the organic solvent to obtain the coating solution of the core-shell structure layer; and
wherein the coating solution of the drug nanoparticle layer is prepared by: mixing the drug nanoparticle with the organic solvent to obtain the coating solution of the drug nanoparticle layer.

30. The method for preparing a drug-coated balloon of claim 29, wherein the organic solvent is at least one selected from the group consisting of alkanes, alcohols and water; and/or, in the coating solution of the core-shell structure layer, a ratio of the added core-shell structure particle by mass to the organic solvent by volume is 2.1 g:3-12 mL; and/or, in the coating solution of the drug nanoparticle layer, a ratio of the added drug nanoparticle by mass to the organic solvent by volume is 150 mg:3-12 mL.
